(19) United States
(12) Patent Application Publication
(10) Pub. No.: US 2021/0193172 A1
(43) Pub. Date: Jun. 24, 2021

(54) INFORMATION PROCESSING SYSTEM, INFORMATION PROCESSING METHOD, AND RECORDING MEDIUM

(71) Applicant: SONY CORPORATION, TOKYO (JP)

(72) Inventor: ATSUSHI SHIONOZAKI, TOKYO (JP)

(21) Appl. No.: 17/250,731

(22) PCT Filed: Aug. 29, 2019
(86) PCT No.: PCT/JP2019/033962

§ 371 Date: Feb. 25, 2021

(30) Foreign Application Priority Data
Sep. 7, 2018 (JP) 2018-168061

(51) Int. Cl. G10L 25/63; G06K 9/00; G06F 9/54

(52) U.S. Cl. CPC G10L 25/63; G06F 9/542; G06K 9/00315

(57) ABSTRACT

An information processing system (1) including a control unit (200) configured to perform control such that, when an emotional condition of a user is detected as being a designated emotional state having a certain amount of separation or more from a normal state and a person who is a factor of the designated emotional state is estimated as being present around the user, people around the user are notified of the designated emotional state.

### TECHNICAL FIELD

The present disclosure relates to an information processing system, an information processing method, and a recording medium.

### BACKGROUND ART

Conventionally, techniques for estimating an emotion of a person based on sensing data have been disclosed.

For example, PTL 1 below discloses a technique for determining whether or not to present an estimated emotion of a user to a plurality of users and controlling presentation based on a determination result thereof.

In addition, PTL 2 below discloses a technique for detecting information representing a physical condition or a mental status of a person in danger, determining a degree of risk, and notifying other persons of a dangerous situation that is unfolding nearby.

### CITATION LIST

### Patent Literature

[PTL 1]

JP 2017-73107A

[PTL 2]

JP 2009-193315A

### SUMMARY

### Technical Problem

However, while there are techniques for conveying an emotion of a user to people around the user, when a factor of the emotion resides in the people around the user, there is no technique for making the people around the user conscious of the fact that they are the factor of the emotion of the user. In addition, while the fact that an emotion belongs to the user is explicitly conveyed to people around the user in PTL 1, in a situation where a large number of strangers go about their lives in densely crowded areas including trains and commercial facilities, letting one's emotions (in particular, unpleasant feelings) be known to people nearby may be undesirable to the user himself/herself.

In consideration thereof, the present disclosure proposes an information processing system, an information processing method and a recording medium capable of making people around an unspecified user conscious of the fact that a factor of an emotion of the user resides in the people around the user.

### Solution to Problem

The present disclosure proposes an information processing system including a control unit configured to perform control so that, when an emotional condition of a user is detected as being a designated emotional state having a certain amount of separation or more from a normal state and a person who is a factor of the designated emotional state is estimated as being present around the user, people around the user are notified of the designated emotional state.

The present disclosure proposes an information processing method including a processor performing control so that, when an emotional condition of a user is detected as being a designated emotional state having a certain amount of separation or more from a normal state and a person who is a factor of the designated emotional state is estimated as being present around the user, people around the user are notified of the designated emotional state.

The present disclosure proposes a recording medium on which is recorded a program for causing a computer to function as a control unit configured to perform control so that, when an emotional condition of a user is detected as being a designated emotional state having a certain amount of separation or more from a normal state and a person who is a factor of the designated emotional state is estimated as being present around the user, people around the user are notified of the designated emotional state.

### Advantageous Effects of Invention

As described above, according to the present disclosure, people around an unspecified user can be made conscious of the fact that a factor of an emotion of the user resides in the people around the user.

It should be noted that the advantageous effect described above is not necessarily restrictive and, in addition to the advantageous effect described above or in place of the advantageous effect described above, any of the advantageous effects described in the present specification or other advantageous effects that can be comprehended from the present specification may be produced.

### BRIEF DESCRIPTION OF DRAWINGS

FIG. 1 is a diagram for explaining an outline of an information processing system according to an embodiment of the present disclosure.

FIG. 2 is a diagram showing an example of an overall configuration of the information processing system according to the present embodiment.

FIG. 3 is a block diagram showing an example of a configuration of an emotion notification server according to the present embodiment.

FIG. 4 is a flow chart showing an example of a flow of basic emotion notification processing according to the present embodiment.

FIG. 5 is a flow chart showing an example of a flow of notification processing of a negative emotion according to the present embodiment.

FIG. 6 is a sequence diagram showing an example of a flow of emotion notification processing in cooperation with an agent system according to the present embodiment.

FIG. 7 is a flow chart showing an example of a flow of notification processing of an emotional change after notification to people nearby according to the present embodiment.

### DESCRIPTION OF EMBODIMENTS

Hereinafter, a preferred embodiment of the present disclosure will be described in detail with reference to the accompanying drawings. In the present specification and the drawings, components having substantially a same functional configuration will be denoted by same reference signs and overlapping descriptions thereof will be omitted.

In addition, descriptions will be given in the following order.

1. Outline of information processing system according to embodiment of present disclosure
2. Configuration example
3. Operation processing
3-1. Emotion notification processing
3-2. Notification processing of negative emotion
3-3. Notification processing in cooperation with agent
3-4. Notification processing of emotional change after notification to people nearby
3-5. Notification processing of positive emotion

### 4. Summary

### 1. Outline of Information Processing System According to Embodiment of Present Disclosure

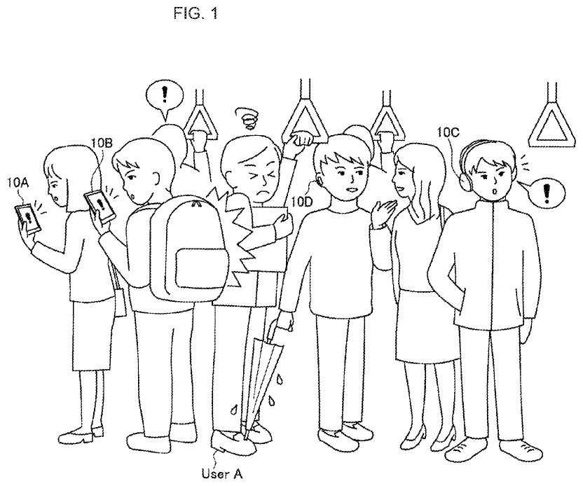

FIG. 1 is a diagram for explaining an outline of an information processing system according to an embodiment of the present disclosure. When the information processing system according to the present embodiment detects an emotion (an emotional condition) of a user A as being a designated emotional state (for example, an unpleasant feeling) having a certain amount of separation or more from a normal state on, for example, a crowded train such as that shown in FIG. 1 and when it is estimated that a factor of the emotion is present among people nearby, the information processing system notifies users around the user A of the detected emotion. For example, when a vibration or a situation indicating that another person's baggage has come into contact with the body of the user A at a timing where the user A develops an unpleasant feeling, it can be estimated that a factor of an emotional change of the user A resides in people around the user A. Although not illustrated, an emotion of the user A can be acquired by, for example, detecting biological information using a wearable device being worn by the user A or analyzing video taken by a surveillance camera on board a train.

Notification to users around the user whose emotion has been detected is favorably performed by a method of subtly making people aware of the fact that there is a person nearby who has developed an unpleasant feeling such as “There is a distressed person around you”, “There is someone around you who is feeling bad”, or “There is someone around you who is bothered by you” without identifying the user whose emotion has been detected or without specifying the factor. Accordingly, users nearby can be made conscious of the fact that their actions may be annoying others and can be prompted to be more mindful. In addition, the person actually feeling annoyed can let those nearby know that he/she is feeling bad without being identified.

Such a notification to users nearby can be performed by various notification terminals 10 (10A to 10D) owned by the respective users including a smartphone terminal, a mobile phone terminal, a tablet terminal, and a wearable device such as smart earphones. Alternatively, the notification terminals 10 may take the form of auditory AR, projection mapping, or the like.

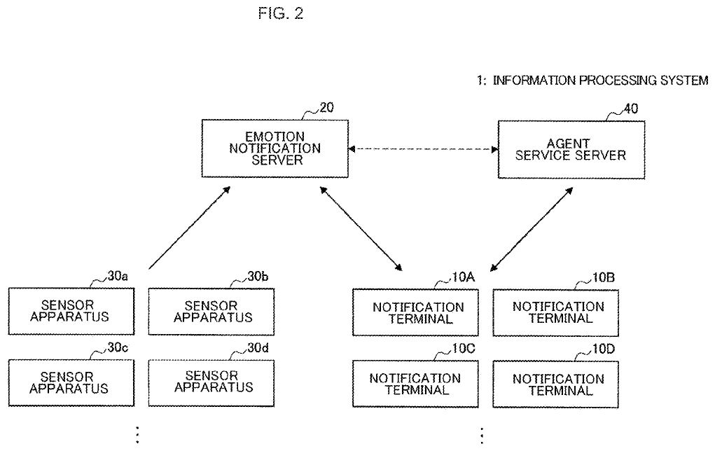

Next, an overall configuration of such an information processing system according to the present embodiment will be described with reference to FIG. 2. FIG. 2 is a diagram showing an example of an overall configuration of the information processing system according to the present embodiment.

As shown in FIG. 2, an information processing system 1 according to the present embodiment includes a sensor apparatus 30, an emotion notification server 20, and a notification terminal 10.

The sensor apparatus 30 may be integrated with the notification terminal 10 or may be provided in the environment as, for example, a surveillance camera, a surveillance microphone, or the like on a train.

The emotion notification server 20 performs emotion estimation based on sensing data acquired from the sensor apparatus 30 and performs notification control to people nearby. The notification to the people nearby is performed using, for example, the notification terminal 10. The notification to the people nearby is not limited to a notification to an individual using an individual terminal and may be a notification by announcement using a public address system.

In addition, the emotion notification server 20 may cooperate with an agent service to realize notification to people nearby via an agent. For example, as shown in FIG. 2, the emotion notification server 20 requests an agent service server 40 to notify users who are present nearby. The agent service server 40 identifies users who are objects from present positional information and the like of the users and causes agents to notify the identified users via the notification terminal 10. Accordingly, since users nearby are informed of the fact that a distressed person is present nearby via an agent which the users utilize on a regular basis and therefore trust, an expected effect is that the notification will likely be genuinely received as a word of advice.

This concludes the description of the information processing system according to the embodiment of the present disclosure. Next, a specific configuration of the emotion notification server 20 included in the information processing system according to the present embodiment will be described with reference to the drawings.

### 2. Configuration Example

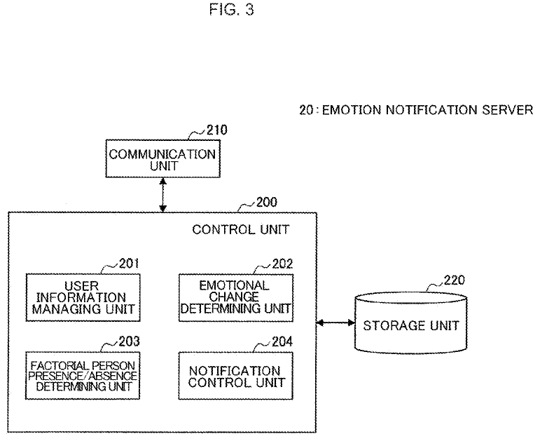

FIG. 3 is a block diagram showing an example of a configuration of the emotion notification server 20 according to the present embodiment. As shown in FIG. 3, the emotion notification server 20 includes a control unit 200, a communication unit 210, and a storage unit 220.

The control unit 200 functions as an arithmetic processing apparatus and a control apparatus and controls whole operations inside the emotion notification server 20 in accordance with various programs. The control unit 200 is realized by an electronic circuit such as a CPU (Central Processing Unit), a microprocessor, or the like. In addition, the control unit 200 may include a ROM (Read Only Memory) for storing programs, operation parameters, and the like to be used and a RAM (Random Access Memory) for temporarily storing parameters and the like that change from time to time.

In addition, the control unit 200 according to the present embodiment also functions as a user information managing unit 201, an emotional change determining unit 202, a factorial person presence/absence determining unit 203, and a notification control unit 204.

The user information managing unit 201 manages information for identifying each user and positional information of the user. The positional information may be managed in absolute coordinates or managed based on information indicating a presence or absence around an object user whose emotion has been detected.

The emotional change determining unit 202 monitors an emotion of a user based on sensing data acquired from the sensor apparatus 30 and comprehends and manages an emotion in a normal state (a usual emotion) of the user which corresponds to a situation of the user. An “emotion” may be expressed by basic emotions such as joy and anger or positiveness/negativeness may be used. The positiveness/negativeness of an emotion corresponds to, for example, a “pleasantness-unpleasantness (valence) of emotion” axis in Russell's circumplex model that organizes human emotions along two axes of “arousal” and “pleasantness-unpleasantness (valence) of emotion”. Examples of positive emotions include happy, elated, excited, relaxed, and satisfied. In addition, examples of negative emotions include upset, angry, discontented, frustrated, unpleasant, sad, depressed, and bored.

In addition, the emotional change determining unit 202 determines whether or not an emotion of the user has changed by a certain amount or more from a normal state which corresponds to a situation of the user. In the present embodiment, for example, when a user develops an unpleasant feeling every time the user boards a crowded train, since the emotion is not caused by a specific nuisance but instead always felt with respect to a crowded situation, a notification to people nearby is not particularly performed. On the other hand, when the user boards a crowded train and an emotion with a higher degree of unpleasantness than usual is suddenly detected, since it is highly likely that the user is suffering some kind of nuisance, a notification to people nearby according to the present embodiment is performed.

The factorial person presence/absence determining unit 203 determines whether or not a person who is a factor of a change in an emotion of the user by a certain amount or more from a normal state is present nearby the user. In the present embodiment, it is not essential to identify a factor or to specify (identify) a person and a determination need only be made that a factor of a change in an emotion of the user by a certain amount or more from a normal state is present outside (nearby) the user instead of within the user. For example, the factorial person presence/absence determining unit 203 can refer to an analysis result of sensing data acquired from the sensor apparatus 30 and determine whether or not a person who is a factor is present nearby based on a nearby situation (or an effect on the user) at the time of change in the emotion of the user such as some kind of object had collided with the user, the object is still in contact with the user, a speaking voice of a person nearby is louder than a threshold, or the like when the user develops an emotion that is more unpleasant than usual.

When the factorial person presence/absence determining unit 203 determines that a factorial person is present nearby, the notification control unit 204 performs a notification for conveying the emotion of the user to an unspecified number of people present around the user. In doing so, favorably, the user is not identified. Even a notification that someone nearby is distressed, someone has developed an unpleasant feeling, or the like is sufficiently conducive to make people nearby more mindful and a person performing the nuisance can be subtly made conscious of the nuisance.

### (Communication Unit 210)

The communication unit 210 is connected to a network in a wired or wireless manner and transmits and receives data to and from the sensor apparatus 30, the notification terminal 10, and the agent service server 40 via the network. For example, the communication unit 210 is communicatively connected to the network by a wired/wireless LAN (Local Area Network), Bluetooth (a registered trademark), or Wi-Fi (Wireless Fidelity (a registered trademark)).

### (Storage Unit 220)

The storage unit 220 is realized by a ROM for storing programs, operation parameters, and the like to be used in processing by the control unit 200 and a RAM for temporarily storing parameters and the like that change from time to time. For example, the storage unit 220 according to the present embodiment accumulates emotions of each user being monitored by the sensor apparatus 30.

This concludes the detailed description of the configuration of the emotion notification server 20 according to the present embodiment. The configuration of the emotion notification server 20 shown in FIG. 3 is merely an example and the present embodiment is not limited thereto. For example, a least a part of components of the emotion notification server 20 may reside in an external apparatus or a least a part of respective functions of the control unit 200 may be realized by the notification terminal 10 or an information processing terminal (for example, a so-called edge server) of which a communication distance is relatively close to the notification terminal 10.

Alternatively, all of the functions of the emotion notification server 20 can be provided in the notification terminal 10 and the present system can be realized solely by the notification terminal 10. In this case, a notification to people nearby may be broadcast (within a prescribed range) from the notification terminal 10 toward nearby notification terminals 10.

### 3. Operation Processing

Next, operation processing of the information processing system according to the present embodiment will be described with specificity with reference to the drawings.

### 3-1. Emotion Notification Processing

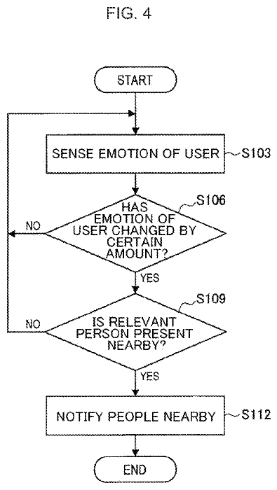

First, basic emotion notification processing according to the present embodiment will be described with reference to FIG. 4. FIG. 4 is a flow chart showing an example of a flow of basic emotion notification processing according to the present embodiment.

As shown in FIG. 4, first, the emotion notification server 20 senses an emotion of the user based on sensing data acquired by the sensor apparatus 30 (step S103). The emotion of the user can be estimated based on biological information of the user (for example, information such as a body temperature, a pulse, a heart rate, perspiration, blinking, brain waves, blinking, or a line of sight), collected voice data, a facial expression, voice information, motion information (a motion, a vibration, or the like of the body that is detected by an acceleration sensor or a gyroscope sensor), environment sensor information (air temperature, humidity, illuminance, wind pressure, or the like), and the like. A specific algorithm for emotion estimation is not particularly limited.

Next, the emotional change determining unit 202 determines whether or not the emotion of the user has changed by a certain amount (from a normal state) (step S106).

Next, the factorial person presence/absence determining unit 203 determines whether or not a relevant person is present around the user (in other words, whether or not a person who was a factor of the emotional change of the user is present nearby the user) (step S109). Whether or not a relevant person is present around the user can be determined such that, when some kind of stimulus is applied to any of the five senses by a person nearby, it is determined that a relevant person is present around the user. In addition, a determination that a factor is present nearby may be made when emotions of a plurality of users at a same location change by a certain amount from a normal state in a same manner.

When it is determined that a relevant person is present around the user (Yes in step S109), the notification control unit 204 notifies people nearby of the emotion of the user (step S112).

Details of each processing step described above will be provided.

### (Change in User's Emotion by Certain Amount)

A change in an emotion of the user by a certain amount indicates that a certain amount of change (either a negative or positive change) has occurred from an emotion in a normal state of the user that is associated with a situation where the user is in. In the present system, a situation where the user is in (a time and date, a location, and circumstances of surroundings) and an emotion in the situation as acquired from sensing data are recorded.

For example, when the user is on a train, a situation where the user is in such as the fact that the user is on a train, duration of the ride, a state of the user (whether the user is seated or standing), crowdedness of the train, and the like is acquired and, at the same time, an emotion of the user (specifically, a value of biological information such as a heart rate or a facial expression of the user may be adopted) in the situation is recorded.

In addition, the emotional change determining unit 202 determines whether or not the emotion of the user changes by a certain amount from a normal state when the user is placed in a situation that has already been recorded. For example, in a case where a heart rate when the crowdedness of the train is 150% is higher by a certain amount or more than a value when riding a train with a normal degree of inclusion (a numeral value in a normal state), the emotional change determining unit 202 determines that a stress level or a degree of frustration of the user has risen (the emotion of the user has changed in a negative direction). Besides the heart rate, a facial expression of the user may be acquired using a camera, and when a frequency of the facial expression being a grimace is higher than when riding a train with a normal degree of inclusion, it is determined that the stress level or the degree of frustration of the user has risen (the emotion of the user has changed in a negative direction).

On the other hand, when the stress level or the degree of frustration of the user rises as the degree of inclusion of the train increases from, for example, 80% to 100%, since it is estimated that the user is feeling unpleasant due to the crowded situation (a reaction in a normal state), a notification to people nearby is not performed. When the stress level or the degree of frustration of the user rises despite the situation such as the degree of inclusion of the train being unchanged, it is estimated that the user is feeling unpleasant due to some factor other than the situation and a notification to people nearby is considered.

The crowdedness on the train can be acquired in cooperation with a cloud service that provides information on crowdedness.

### (Whether or Not a Relevant Person is Nearby)

The factorial person presence/absence determining unit 203 first determines, based on sensing data, whether or not there has been a change in a stimulus to the five senses of the user such as an occurrence of a sound or an occurrence of an odor nearby or the presence or absence of contact in temporal synchronization with a change in the emotion of the user.

Conceivable examples of the occurrence of a sound on a train include sound leaking from someone's headphones and snoring. Such sounds can be detected by a microphone provided in a device belonging to the user or by a microphone provided in the environment.

The occurrence of an odor can be detected by an odor sensor. The odor sensor is also conceivably provided in a device belonging to the user or provided in the environment. For example, odors such as an extremely strong smell of tobacco, body odor, and the smell of food or beverages can be detected.

The presence or absence of contact can be determined based on, for example, detection of a sudden motion or vibration of the user. Regarding a motion of the body or the like, a contact/impact received by an upper body, a lower body, a right side of the body, or a left side of the body can be sensed by an acceleration sensor or a gyroscope sensor provided in one or more wearable devices being worn on the body.

When a stimulus to the five senses such as a sound, an odor, or contact is detected in temporal synchronization with a change in the emotion of the user, the factorial person presence/absence determining unit 203 determines that a relevant person is present around the user (a person who is a factor is present nearby).

When it is difficult to accurately determine a sound, an odor, contact, or the like (when sensing data is unclear, insufficient, or the like), for example, a cause of the change in emotion of the user may be explored by asking the user appropriate questions through an agent being used by the user. For example, the cause may be confirmed by asking a question such as “Did something come into contact with you?”, “Do you hear something that is annoying you?”, or “Do you smell something that is annoying you?”.

### (Notification to People Nearby)

When issuing a notification to people nearby, the notification control unit 204 may determine an extent (in terms of distance) of a range in which one or more persons to be or not to be issued a notification are present or may issue a notification to people present within a range set in advance.

For example, when a position of the user or people nearby can be estimated with high accuracy, a range with a radius of 1 m may be determined in a case where contact is made, a range with a radius of 2 m may be determined in a case of an odor, and a range may be determined in a case of sound in accordance with the type of sound (a range with a radius of 1 m in a case of headphones and a range with a radius of 2 m in a case of snoring). In addition, when a direction or a position of an occurrence of a factor can be estimated such as a contact from the right, a sound from the left (an approximate position of a sound source can be specified using a directional microphone), or a position and/or a direction of an odor generation source, a notification may be issued to people present in the direction or around the position. In the present embodiment, it is not essential to identify a person having generated the factor and by notifying people around the user (at least people nearby the factor) of the emotion of the user, subtly making people nearby conscious of the emotion of the user will suffice.

Furthermore, as the notification to people nearby, a push notification or the like using a smartphone or a mobile phone terminal, a tablet terminal, or a wearable device such as smart earphones or a smart headset being used by each person may be adopted. In addition, the emotion notification server 20 may cooperate with a system of an environment in which the user is present and perform a notification in the form of, for example, an announcement using a public address system or digital signage.

Contents of the notification favorably subtly represent an emotional condition of the user. For example, by conveying the emotional condition of the user by a notification reading “There is a frustrated person nearby. Do you have any idea why this person is frustrated?” or “There is a person who is distressed because of a nuisance”, people nearby can be subtly made conscious of the fact that his or her own action constitutes a nuisance. In addition, when a specific factor is identified, people nearby may be notified of direct contents such as “Be mindful of sound leaking from your headphones”, “Be mindful of the odor of tobacco”, or “Be mindful of how you carry your baggage”. Furthermore, an indirect warning notification such as “The train is getting crowded, so be mindful that your hand baggage does not get in the way of others” may be performed. When a person who is a factor is identified (for example, when the factor is an “umbrella”, a person nearby who is carrying an umbrella is identified), the person may be notified of direct contents such as “Be mindful of how you carry your umbrella. There is someone who is frustrated about being struck by the umbrella.”

### 3-2. Notification Processing of Negative Emotion

Next, as an example of an emotional change of the user, a case where an emotion changes by a certain amount in a negative direction from a normal state will be described.

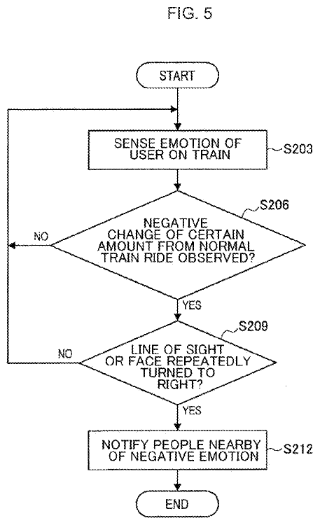

FIG. 5 is a flow chart showing an example of a flow of notification processing of a negative emotion according to the present embodiment. A case where the user is on a train will be assumed as an example.

As shown in FIG. 5, first, the sensor apparatus 30 senses an emotion of the user on the train (step S203). Sensing data is sent to the emotion notification server 20 and accumulated in the storage unit 220. In the storage unit 220, the emotion is accumulated in association with a situation where the user is in as a monitoring result of the emotion of the user. The accumulated emotion may be a negative or positive value (a calculation result), a value of sensing data such as a heart rate, or a value of a specific emotion such as angry, sad, or happy.

Next, the emotional change determining unit 202 determines whether or not an abrupt change in emotion by a certain amount in the negative direction as compared to when the user is usually riding the train has been observed (step S206). An emotion in the negative direction (a degree of frustration or the like) can be determined by detecting a fluctuation in the heart rate of the user, sighing, frustrated muttering, grimacing, or the like.

Next, the factorial person presence/absence determining unit 203 determines whether or not the line of sight or the face has repeatedly turned to the right (step S209). In this case, as an example of determining whether or not a factor of the emotional change of the user is present nearby based on a movement of the line of sight or the face of the user, a determination of “whether or not the line of sight or the face has repeatedly turned to the right (or to the left; in any case, at least in one direction)” is made. For example, when the line of sight of the user of which an emotional change has been detected is frequently turned to the right or when the face is also repeatedly turned in a same direction, the factorial person presence/absence determining unit 203 can determine that the factor is present nearby. When the user is not cautious of his/her surroundings such as when the user is not facing a particular direction or the user's gaze is fixed on a screen of a smartphone, a determination can be made that the factor of the emotional change is not present nearby.

In addition, the factorial person presence/absence determining unit 203 notifies people nearby of the negative emotion (the fact that there is a user with the negative emotion) (step S212). When positions of people nearby and the user can be accurately determined, the factorial person presence/absence determining unit 203 may only notify people present in the direction in which the line of sight or the face of the user has repeatedly turned (a direction which the user is concerned about).

Details of each processing step described above will be provided.

### (Change in Emotion of User by Certain Amount)

For example, in a situation where the user is on a train, the fact that the user is in a different emotional condition than a normal state is detected based on a change in the heart rate (a 10% increase or the like), the presence or absence of frustrated mumbling in the form of self-talk (such as three times or more), recognition of the face or a facial expression of the user captured through a front camera of a smartphone or the like (an increase (such as a 10% increase) in a degree of grimacing or the presence or absence of grimacing), or the like.

### (Determination of Presence/Absence of Factorial Person Based on Line of Sight or the Like of User)

A movement of a line of sight or the face of the user can be performed by, for example, the front camera of a smartphone owned by the user, a camera or various sensors (such as an acceleration sensor and a gyroscope sensor) provided in smart eyeglasses being worn by the user, or the like. When it is observed that the line of sight of the user is oriented in another direction for a certain period of time at a same timing as a change in emotion instead of being directed toward the front or towards the hands (specifically, a screen of a smartphone, a book or a newspaper being read by the user, or the like), it can be determined that some kind of factor of the emotional change is present in that direction. In addition, when it is observed that one or more persons around the user also turn their heads in a same direction at a same timing as the change in emotion of the user, it can be determined that some kind of factor (a person) (who is of interest to a large number of people) is present in that direction.

### (Notification to People Nearby)

For example, based on positional information of the user, the notification control unit 204 issues a notification to one or more persons present in a predetermined threshold range (for example, within 2 m) in a direction in which a factor is estimated to be present. When the presence or absence of the factor has not been determined, propriety of a notification may be confirmed with the user.

### 3-3. Notification Processing in Cooperation with Agent

Next, a case where the emotion notification server 20 cooperates with an agent system and issues a notification to people nearby via an agent will be described.

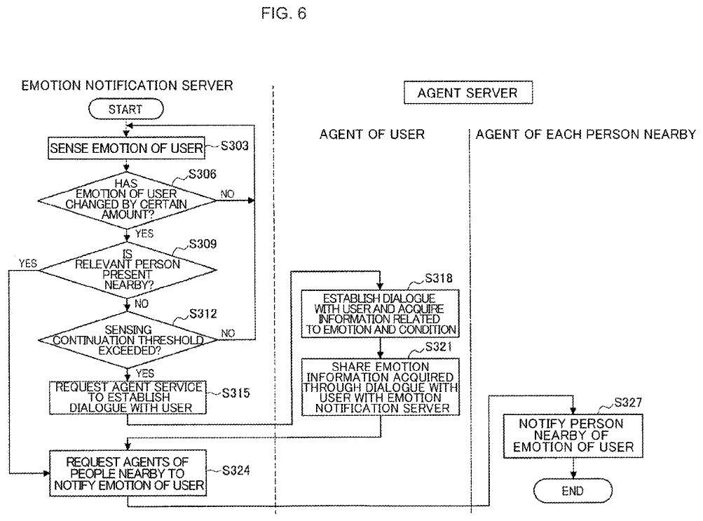

FIG. 6 is a sequence diagram showing an example of a flow of emotion notification processing in cooperation with an agent system according to the present embodiment.

As shown in FIG. 6, first, the emotion notification server 20 senses an emotion of the user (step S303) and determines whether or not the emotion of the user has changed by a certain amount (step S306).

Next, a determination is made as to whether or not a relevant user is present nearby (step S309).

The steps S303 to S312 are similar to the processing of the embodiment described with reference to FIGS. 4 and 5.

Next, in a case where whether or not a relevant user is present nearby could not be determined (No in step S309), when a threshold (timeout) for continuing sensing is exceeded (Yes in step S312), the emotion notification server 20 requests the agent service to establish a dialogue with the user (step S315). In other words, in a case where whether or not a relevant user is present nearby is unclear (for example, due to unclear or insufficient sensing data), when a determination cannot be made even after performing sensing for a certain amount of time, the emotion notification server 20 may cooperate with the agent service and find out about a factor (details of an emotion or a condition of the user) from the user through a dialogue with an agent.

The agent service server 40 establishes a dialogue with the user using the agent of the user (for example, via a smartphone or a smart earphone being used by the user) and acquires information on an emotion or a condition (step S318). Specifically, for example, the agent asks the user a question like “What's the matter?”, “Why are you frustrated?”, or “Was something annoying done to you?”.

Next, the agent service server 40 shares emotion information acquired through the dialogue with the user with the emotion notification server 20 (step S321).

Next, the emotion notification server 20 requests the agent service server 40 to notify agents of people nearby the user of the emotion of the user (step S324).

In addition, the agent service server 40 performs control so that people present around the user (as a premise, limited to people using the agent system) are notified by the agents used by the people of the emotion of the user (step S327). For example, “There is a frustrated person nearby” is conveyed by a voice of the agents. Since the notification is issued via agents which the people nearby utilize on a regular basis and therefore trust, an effect that the notification will likely be genuinely received as a word of advice can be expected.

In the case of the present embodiment, user identification information is favorably associated (or unified) in advance between the emotion notification system (the emotion notification server 20) and the agent service system (the agent service server 40). Alternatively, people may be associated using a public authentication or identification system. In other words, a “user of which an emotional change has been detected” and users determined as “people nearby” must be associated by the emotion notification system with user identification information in the agent system. Alternatively, the emotion notification system may delegate user management to the agent service system.

In addition, while a request for a notification via agents to people nearby is made from the emotion notification server 20 to the agent service server 40 in the sequence diagram shown in FIG. 6, the present embodiment is not limited thereto and a request for a notification may be made from the agent of the user of which an emotional change has been detected to the agents of the people nearby.

### 3-4. Notification Processing of Emotional Change After Notification to People Nearby

Next, notification processing of an emotional change after a notification to people nearby will be described. Once an emotional change (in particular, a negative emotion) of the user is conveyed to people nearby, the people nearby may conceivably become conscious of an action or a state that is a factor and correct the action or the state. In this case, since it is expected that the correction of the action or the state that is the factor will cause the negative emotion of the user to change to a positive emotion (or at least reduce the emotion in the negative direction), the people nearby may be notified of such a change together with a word of gratitude. Accordingly, a person can be made conscious of the fact that the cause was the person's own action after all, and by conveying gratitude instead of simply telling people who were not the factor to be mindful, it is expected that an aftertaste of the experience will be improved.

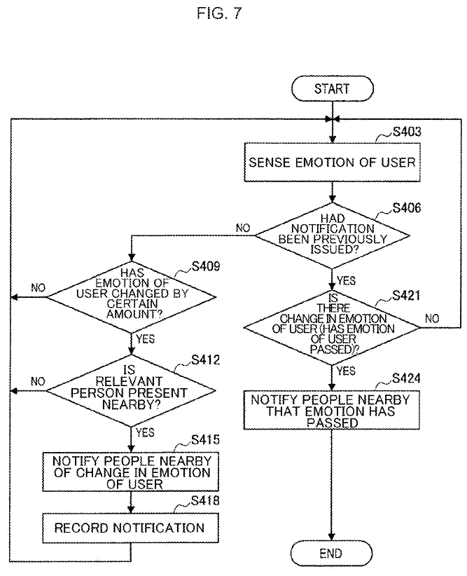

FIG. 7 is a flow chart showing an example of a flow of notification processing of an emotional change after a notification to people nearby according to the present embodiment.

As shown in FIG. 7, first, the sensor apparatus 30 continuously performs emotion sensing of the user (step S403). Sensing data is sent from the sensor apparatus 30 to the emotion notification server 20.

Next, whether or not a previous notification had been issued is confirmed (step S406). The confirmation of a “previous notification” may be a confirmation of a notification within a prescribed period of time in the past.

Next, when a previous notification had not been issued (No in step S406), processing described in steps 409 to S418 is performed. Since the processing described in steps 409 to S418 is similar to the respective steps of notification processing described with reference to FIGS. 4 to 6, details will be omitted here.

On the other hand, when a previous notification had been issued (Yes in step S406), the emotional change determining unit 202 of the emotion notification server 20 determines whether or not a change has occurred in an emotion of the user (step S421).

In addition, people nearby are notified of the fact that the emotion has passed (step S424). In other words, in the processing described in steps 409 to S418, when a notification of the emotion of the user is issued to people nearby and, subsequently, the factor is corrected and the emotion of the user changes once again (specifically, a negative emotion passes or the like), the notification control unit 204 once again notifies the people nearby in a similar manner that the emotion of the user has passed. In doing so, the notification control unit 204 may issue the notification together with a word of gratitude.

Even in this case, by cooperating with an agent service system, the notification may be issued from each agent. For example, each agent of the people nearby may convey a message such as “It looks like the frustration has passed! They even thanked me . . . ”.

In addition, when a person nearby changes his/her action after receiving the previous notification and subsequently receives a notification such as “It seems like the frustrated feeling has settled down. Thank you.”, the person can be made conscious of the fact that his/her own action had been the cause of the frustration.

### 3-5. Notification Processing of Positive Emotion

While cases where an emotion of a user changes in a negative direction have been mainly explained by presenting specific examples in each embodiment described above, the present embodiment is not limited to cases where the emotion of the user changes in the negative direction and people around the user (or at least people nearby including a person who is a factor) can be notified of an emotion (positive) of the user when the emotion of the user changes in a positive direction.

For example, a case will be considered where a happy feeling of a user (a customer) is enhanced by service provided by a server or a sales clerk at a restaurant or a store.

Generally, when a server or a sales clerk serves a customer at a restaurant or a store, timings at which the server or the sales clerk interacts with a customer or a frequency of such interactions may elicit a positive or negative emotion of the customer. For example, if the customer is addressed by the sales clerk when the customer is unsure of which item to purchase, a happy and positive emotion such as “Thank you for noticing!” may conceivably be invoked. On the other hand, if the customer is addressed when the customer does not want to be bothered such as when the customer desires to do “solo-shopping”, a negative emotion such as “Ugh!” may conceivably be invoked.

Since it is difficult for the sales clerk to accurately assess such a state (a state of mind) of the customer in advance, by detecting and notifying a change in the emotion of the customer after serving the customer, the sales clerk can be given encouragement or made conscious.

For example, when a timing at which the sales clerk addresses the customer causes a positive emotional change in the customer, for example, the emotion notification server 20 issues a notification such as “The customer seems satisfied” to sales clerks inside the store (at least including the sales clerk having addressed the customer). Even when the customer had exited the store without making a purchase, the fact that the customer had exited the store in a good mood (with a happy feeling represented by, for example, an increase in a degree of smiling) because the sales clerk had addressed the customer is conveyed to the sales clerk.

In addition, a change in the emotion of the user (the customer) can be detected from a change in the degree of smiling, a tone of conversation, utterances, or the like.

Furthermore, whether or not a person (a sales clerk) to be a factor is present nearby may be determined based on, for example, contents of an utterance by the user (a tone in which “thank you” is said to the sales clerk or a conversation such as “I really liked your recommendation”) or the like.

Notifying a sales clerk who is an object, sales clerks nearby, or people (other customers) nearby other than or in addition to sales clerks of the fact that the customer has a positive emotion (for example, by saying that “There is a customer who feels being addressed was very helpful”, “There is someone who is happier”, or the like) encourages the sales clerks and, with respect to other customers, it is expected that their impressions of the store will improve and make them consider starting a conversation with a sales clerk.

In addition, by expanding a notification range to the outside of the store, the notification can also be issued to potential customers who are present around the store. Accordingly, interest toward the store can be aroused.

Furthermore, an example of a factor that stimulates the five senses of a user at a timing where the emotion of the user changes in a positive direction is not limited to being addressed by a sales clerk as described above and, for example, an aroma or a sound may be assumed. For example, it can also be considered that a pleasant aroma (an aroma that is generally favored such as that of a flower, a fruit, soap, shampoo, or perfume), a pleasant piece of music, or the like has been created around the user at a timing where the emotion of the user had changed in a positive direction. Detection of the aroma or the sound can be performed by a (specific) aroma sensor or a microphone.

In this case, the emotion notification server 20 may issue a subtle notification such as “There is someone who has become happy” or a specific notification directly linked to the aroma such as “Someone nearby seems to be happy because of the pleasant aroma/the pleasant sound” to people nearby.

### 4. Summary

As described above, the information processing system according to the embodiment of the present disclosure enables people around an unspecified user to be made conscious of the fact that a factor of an emotion of the user resides in the people around the user.

While a preferred embodiment of the present disclosure has been described in detail with reference to the accompanying drawings, the present technique is not limited thereto. It will be obvious to a person with ordinary skill in the art to which the technical field of the present disclosure pertains that various modifications and changes can be arrived at without departing from the scope of the technical ideas as set forth in the appended claims and, as such, it is to be understood that such modifications and changes are to be naturally covered in the technical scope of the present disclosure.

For example, a computer program can also be created which causes hardware such as a CPU, a ROM, and a RAM that are built into the notification terminal 10 or the emotion notification server 20 described above to fulfill functions of the notification terminal 10 or the emotion notification server 20. In addition, a computer-readable storage medium storing the computer program is also provided.

Furthermore, detection of an emotional change is not limited to a single user and, when a similar emotional change of a plurality of users is detected, contents of the emotional change may be notified. For example, a notification such as “There seems to be many frustrated people around you” may be considered.

In addition, while an example of each step of operation processing according to the present embodiment has been described with reference to FIGS. 4 to 7, the operation processing shown in FIGS. 4 to 7 is simply an example and the present disclosure is not limited to the example shown in FIGS. 4 to 7. For example, the present disclosure is not limited to an order of steps shown in FIGS. 4 to 7. At least any of the steps may be processed in parallel or processed in reverse order. Furthermore, all of the processing steps shown in FIGS. 4 to 7 need not necessarily be executed. Moreover, all of the processing steps shown in FIGS. 4 to 7 need not necessarily be performed by a single apparatus.

Furthermore, the advantageous effects described in the present specification are merely descriptive or exemplary and not restrictive. In other words, the technique according to the present disclosure can produce, in addition to or in place of the advantageous effects described above, other advantageous effects that will obviously occur to those skilled in the art from the description of the present specification.

The present technique can also be configured as follows.

(1)

An information processing system including a control unit configured to perform control such that, when an emotional condition of a user is detected as being a designated emotional state having a certain amount of separation or more from a normal state and a person who is a factor of the designated emotional state is estimated as being present around the user, people around the user are notified of the designated emotional state.

(2)

The information processing system according to (1), wherein the control unit is configured to

detect the emotional condition in accordance with any of biological information, a facial expression, and voice information of the user.

(3)

The information processing system according to (1) or (2), wherein the control unit is configured to

detect that the user is in the designated emotional state at a certain frustration level or higher in accordance with any of a heart rate, a facial expression, and contents of mumbling of the user.

(4)

The information processing system according to (1) or (2), wherein the control unit is configured to

detect that the user is in the designated emotional state as an emotion with a certain level of positiveness or higher in accordance with any of a degree of smiling, a tone of a conversation, and contents of the conversation of the user.

(5)

The information processing system according to any one of (1) to (4), wherein the control unit is configured to

estimate that a person who is a factor of the designated emotional state is present nearby when there is a change in sensor data acquired by any of a microphone, an image sensor, a contact sensor, and an odor sensor in synchronization with a change in the emotional condition.

(6)

The information processing system according to any one of (1) to (5), wherein the control unit is configured to

estimate that a person who is a factor of the designated emotional state is present nearby when it is detected that the user is facing a same direction for a certain period of time in synchronization with a change in the emotional condition.

(7)

The information processing system according to any one of (1) to (6), wherein the control unit is configured to

estimate that a person who is a factor of the designated emotional state is present nearby when it is detected that the user and people nearby are facing a same direction for a certain period of time in synchronization with a change in the emotional condition.

(8)

The information processing system according to any one of (1) to (7), wherein the control unit is configured to

request an agent of an agent service being used by the user or the agent service to notify agents of people nearby of the designated emotional state.

(9)

The information processing system according to (8), wherein the control unit is configured to

estimate that a person who is a factor of the designated emotional state is present nearby based on contents of a dialogue between the user and the agent.

(10)

The information processing system according to (9), wherein the control unit is configured to

estimate that a person who is a factor of the designated emotional state is present nearby based on contents of answers provided by the user which are obtained by having the agent ask questions to the user about a factor of the designated emotional state.

(11)

The information processing system according to any one of (1) to (10), wherein the control unit is configured to,

after requesting an agent of an agent service being used by the user or the agent service to notify agents of people nearby of the designated emotional state and when a return of the user to an emotional condition in a normal state is detected, request the agent of the agent service being used by the user or the agent service to notify the agents of people nearby of the return of the designated emotional state to an emotional condition in a normal state.

(12)

An information processing method including

a processor

performing control such that, when an emotional condition of a user is detected as being a designated emotional state having a certain amount of separation or more from a normal state and a person who is a factor of the designated emotional state is estimated as being present around the user, people around the user are notified of the designated emotional state.

(13)

A recording medium on which is recorded a program for

causing a computer to function as

a control unit configured to perform control such that, when an emotional condition of a user is detected as being a designated emotional state having a certain amount of separation or more from a normal state and a person who is a factor of the designated emotional state is estimated as being present around the user, people around the user are notified of the designated emotional state.

### REFERENCE SIGNS LIST

1 Information processing system
10 Notification terminal
20 Emotion notification server
30 Sensor apparatus
40 Agent service server
200 Control unit
201 User information managing unit
202 Emotional change determining unit
203 Factorial person presence/absence determining unit
204 Notification control unit
210 Communication unit
220 Storage unit

1. An information processing system comprising a control unit configured to perform control such that, when an emotional condition of a user is detected as being a designated emotional state having a certain amount of separation or more from a normal state and a person who is a factor of the designated emotional state is estimated as being present around the user, people around the user are notified of the designated emotional state.

2. The information processing system according to claim 1, wherein the control unit is configured to
detect the emotional condition in accordance with any of biological information, a facial expression, and voice information of the user.

3. The information processing system according to claim 1, wherein the control unit is configured to
detect that the user is in the designated emotional state at a certain frustration level or higher in accordance with any of a heart rate, a facial expression, and contents of mumbling of the user.

4. The information processing system according to claim 1, wherein the control unit is configured to
detect that the user is in the designated emotional state as an emotion with a certain level of positiveness or higher in accordance with any of a degree of smiling, a tone of a conversation, and contents of the conversation of the user.

5. The information processing system according to claim 1, wherein the control unit is configured to
estimate that a person who is a factor of the designated emotional state is present nearby when there is a change in sensor data acquired by any of a microphone, an image sensor, a contact sensor, and an odor sensor in synchronization with a change in the emotional condition.

6. The information processing system according to claim 1, wherein the control unit is configured to
estimate that a person who is a factor of the designated emotional state is present nearby when it is detected that the user is facing a same direction for a certain period of time in synchronization with a change in the emotional condition.

7. The information processing system according to claim 1, wherein the control unit is configured to
estimate that a person who is a factor of the designated emotional state is present nearby when it is detected that the user and people nearby are facing a same direction for a certain period of time in synchronization with a change in the emotional condition.

8. The information processing system according to claim 1, wherein the control unit is configured to
request an agent of an agent service being used by the user or the agent service to notify agents of people nearby of the designated emotional state.

9. The information processing system according to claim 8, wherein the control unit is configured to
estimate that a person who is a factor of the designated emotional state is present nearby based on contents of a dialogue between the user and the agent.

10. The information processing system according to claim 9, wherein the control unit is configured to
estimate that a person who is a factor of the designated emotional state is present nearby based on contents of answers provided by the user which are obtained by having the agent ask questions to the user about a factor of the designated emotional state.

11. The information processing system according to claim 1, wherein the control unit is configured to
after requesting an agent of an agent service being used by the user or the agent service to notify agents of people nearby of the designated emotional state and when a return of the user to an emotional condition in a normal state is detected, request the agent of the agent service being used by the user or the agent service to notify the agents of people nearby of the return of the designated emotional state to an emotional condition in a normal state.

12. An information processing method comprising
a processor
performing control such that, when an emotional condition of a user is detected as being a designated emotional state having a certain amount of separation or more from a normal state and a person who is a factor of the designated emotional state is estimated as being present around the user, people around the user are notified of the designated emotional state.

13. A recording medium on which is recorded a program for causing
a computer to function as
a control unit configured to perform control such that, when an emotional condition of a user is detected as being a designated emotional state having a certain amount of separation or more from a normal state and a person who is a factor of the designated emotional state is estimated as being present around the user, people around the user are notified of the designated emotional state.
